(19) United States
(12) Patent Application Publication
(10) Pub. No.: US 2023/0073829 A1
(43) Pub. Date: Mar. 9, 2023

(54) TIBIAL SIZING GUIDE

(71) Applicants: Robert Miller Carlisle, II, Sandusky, OH (US); Scott Edward Pehelps, Garrettsville, OH (US)

(72) Inventors: Robert Miller Carlisle, II, Sandusky, OH (US); Scott Edward Phelps, Garrettsville, OH (US)

(21) Appl. No.: 17/895,451

(22) Filed: Aug. 25, 2022

Related U.S. Application Data
(60) Provisional application No. 63/240,511, filed on Sep. 3, 2021.

(51) Int. Cl. A61B 17/15

(52) U.S. Cl. CPC A61B 17/157

(57) ABSTRACT

An assembly for sizing a tibia includes a plurality of tibial sizing guides. Each of the tibial sizing guides includes a tibial sizing template. The tibial sizing templates are each of different dimensions to accommodate different sizes of cut tibia surfaces.

This application claims the benefit of U.S. Provisional Application Ser. No. 63/240,511, filed Sep. 3, 2021, the disclosure of which is incorporated herein by reference in its entirety.

### BACKGROUND

The exemplary embodiment relates to joint replacement and, in particular, to an assembly of tibial sizing guides suited to use in selection of a suitable tibial replacement component selected from a set of standard-sized tibial replacement components.

Osteoarthritis affects millions of people worldwide. It is one of the leading causes for pain and disability in the United States. (See, for example, Nigel Arden, et al., “Osteoarthritis: Epidemiology, Best Practice & Research Clinical Rheumatology,” Volume 20, Issue 1, pp. 3-25 (2006)). Generally, arthritis is the result of breakdown or degradation of cartilage within a joint. A joint is the articulation formed between ends of two separate bones and cartilage is a connective tissue composed of collagen and other proteins adhered to ends of each bone that provides a smooth surface for cushioning and articulation within a joint.

Arthritis can occur in any joint within the body. One of the most common sites is the knee. Similar to arthritis treatments for other joints, conservative treatments for knee arthritis may include physical therapy, non-steroidal anti-inflammatory drugs (NSAIDs), injections within the joint with steroids, viscosupplementation, and combinations thereof. A goal of treatment is to alleviate pain so that patients can regain their function and return to an active lifestyle. However, when conservative management fails to achieve these goals, surgery may be considered.

A total knee replacement (TKR) surgery generally removes the arthritis within the knee joint and replaces the bony surfaces with metal and plastic components. Total knee replacement has proved to be very successful at alleviating pain, restoring function, and improving quality of life. It is anticipated that with an aging population, the prevalence of osteoarthritis will only increase and subsequently the number of TKRs could reach 1 million per year by 2030, in the U.S. (See, Sloan et al., “Projected Volume of Primary Total Joint Arthroplasty in the U.S., 2014 to 2030,” J. Bone Joint Surg. Am. 100(17):1455-60 (2018)).

In performing a TKR surgery, damaged surface of the femur (thigh bone), tibia (shin bone), and patella (kneecap) are removed. Each bone is sized to select suitably-sized implants to ensure balance within the knee for appropriate stability and motion. The selected implants are then positioned and held in place with bone cement. Various methods exist for tibial sizing. Commonly, a handle attached to a tibial sizing plate is placed on the cut tibial surface to find the appropriate size for the necessary tibial component rotation. One example of this type is the Persona™ tibial sizing plate available from Zimmer Biomet (see, https://www.zimmerbiomet.com/content/dam/zimmer-biomet/medical-professionals/knee/persona-knee-system/persona-the-personalized-knee-surgical-technique.pdf, page 24, FIGS. 49a, 49b, and 50, hereinafter, Zimmer Biomet catalog). To determine the correct size, an initial tibial plate size, attached to the handle, is estimated by surgical staff and handed to the surgeon. The surgeon evaluates this initial tibial plate size on the cut tibial surface for appropriate fit and rotation. In the event the surgeon would like to evaluate a different tibial plate size, a different sized tibial plate is taken from the sterile storage tray on the surgical field and attached to the handle. This process is repeated until the surgeon finds the appropriate fit and rotation. Once a size has been established, the tibial sizing plate is secured to the tibial surface with multiple screws. This allows trialing and further preparation of the tibia to accommodate the final implant(s).

There are various disadvantages to this approach. It is time consuming to fit each sizing plate to the handle, taking up valuable time in the surgery. Additionally, at the end of the procedure, the handle, all the sizing plates, and the storage tray need to be sterilized and sealed in a sterile pouch for subsequent reuse.

There remains a need for a more efficient system and method for sizing a bone, such as a tibia, during surgery.

### BRIEF DESCRIPTION

In accordance with one aspect of the exemplary embodiment, an assembly includes a plurality of tibial sizing guides, each of the tibial sizing guides comprising a tibial sizing template, each tibial sizing template being of different dimensions.

In accordance with another aspect of the exemplary embodiment, a method of sizing a tibia surface includes providing an assembly as described herein, placing one of the tibial sizing guides on a cut surface of a tibia, placing another of the tibial sizing guides on the cut surface of the tibia, identifying one of the placed tibial sizing guides as being a best fit for the tibia surface, identifying a tibial component corresponding to the identified one of the placed tibial sizing guides from a plurality of tibial components of different dimensions, and fitting the identified tibial component to the tibia surface.

In accordance with another aspect of the exemplary embodiment, a tibial sizing assembly includes a plurality of tibial sizing guides. Each of the tibial sizing guides includes a handle connected to a tibial sizing template. Each tibial sizing template is of different dimensions from the other(s). Each tibial sizing template includes first and second lobes of different dimensions to each other. The handle defines a hole. The tibial sizing guides are attached to a ring by their respective holes.

### BRIEF DESCRIPTION OF THE DRAWINGS

FIG. 1 is a perspective view of a tibial sizing assembly in accordance with a first aspect of the exemplary embodiment;

FIG. 2 is another perspective view of the tibial sizing assembly of FIG. 1;

FIG. 3 is a perspective view of a first side of a first tibial sizing guide (sizing guide A) of the assembly of FIG. 1;

FIG. 4 is a perspective view of a second side of the first tibial sizing guide of the assembly of FIG. 1;

FIG. 5 is a perspective view of a first side of a second tibial sizing guide (sizing guide B) of the assembly of FIG. 1;

FIG. 6 is a perspective view of a second side of the second tibial sizing guide of the assembly of FIG. 1;

FIG. 7 is a perspective view of a first side of a third tibial sizing guide (sizing guide C) of the assembly of FIG. 1;

FIG. 8 is a perspective view of a second side of the third tibial sizing guide of the assembly of FIG. 1;

FIG. 9 is a perspective view of a ring of the assembly of FIG. 1;

FIGS. 10A, 10B, 10C, 10D, and 10E illustrate positioning the sizing guide on a right tibia bone; and

FIG. 11 illustrates a method of use of the assembly of FIG. 1.

### DETAILED DESCRIPTION

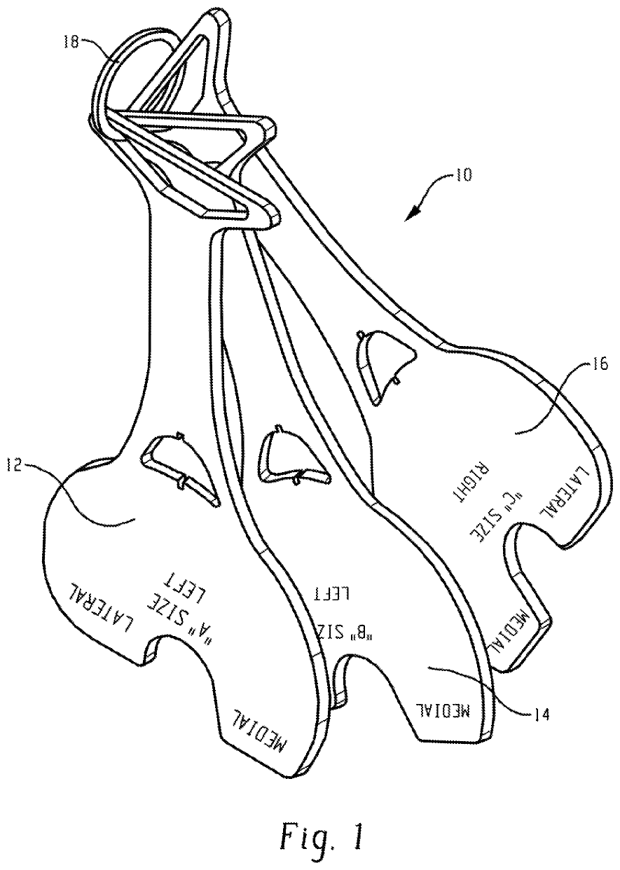

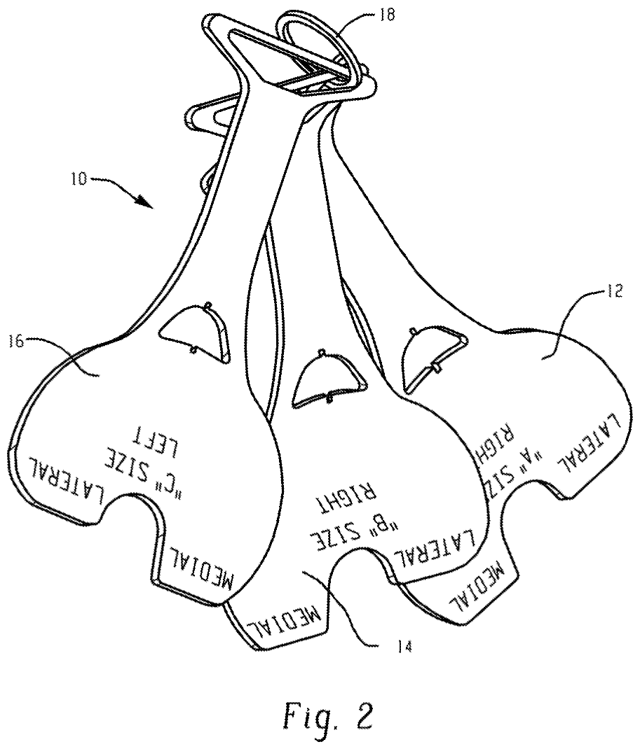

With reference to FIGS. 1 and 2, a tibial sizing guide assembly 10 is shown in perspective view. The assembly includes a set of tibial sizing guides 12, 14, 16. In the illustrated embodiment, there are three tibial sizing guides, although fewer or more sizing guides may be included, such as from 2 to 10 or from 3 to 6. In general, the number of tibial sizing guides corresponds to at least the number of different sized tibial replacement plates (or other tibial components) available to be selected for use in a total knee replacement surgery.

The tibial sizing guides 12, 14, 16 may be interconnected. For example, tibial sizing guides 12, 14, 16 are moveably and removably connected by a ring 18, similar to a key ring, to which the sizing guides can be attached and removed. The ring has first and second ends for purposes of attaching the tibial sizing guides. This allows for different numbers and/or sizes of sizing guides to be provided in the assembly. It also allows for easy sterilization of the sizing guides. In other embodiments, the set of sizing guides may be simply provided in a sterilized pouch or otherwise connected or collected together.

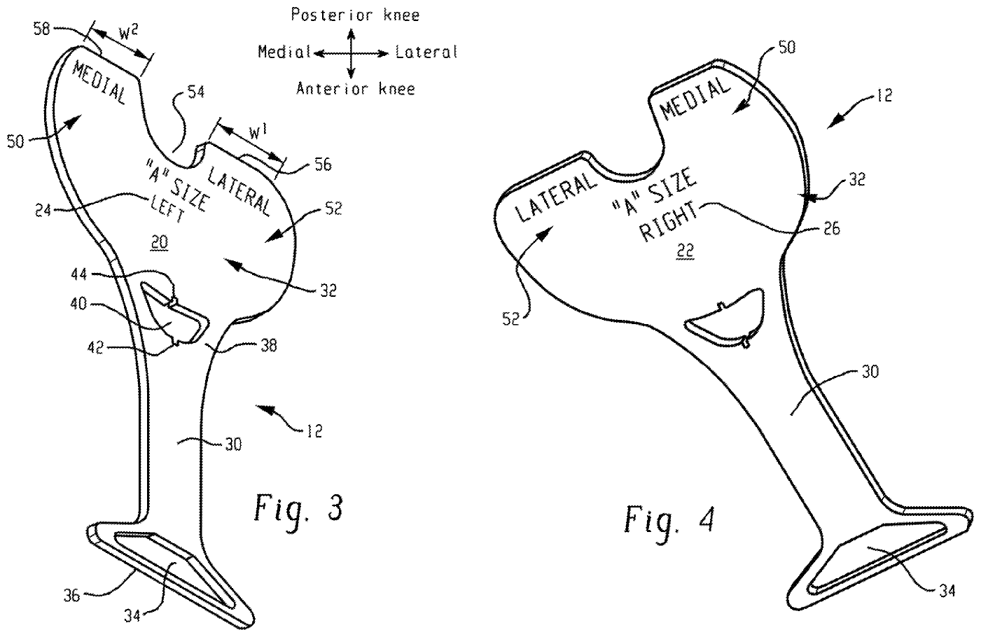

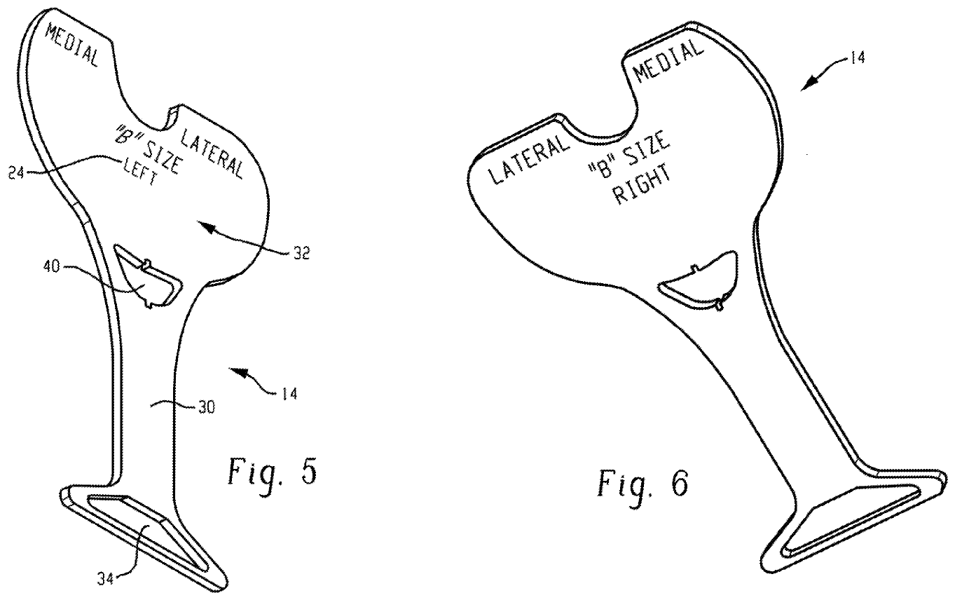

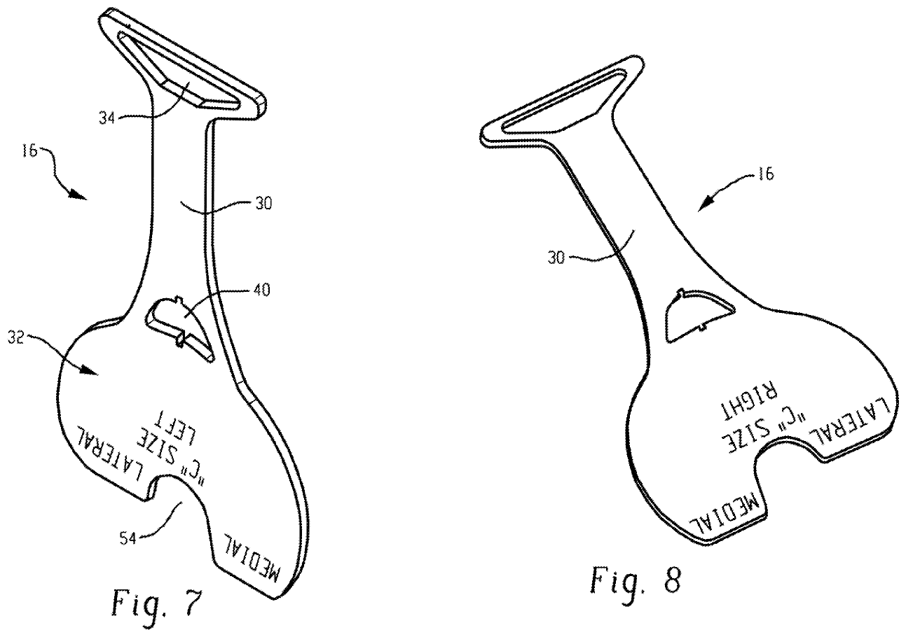

With reference to FIGS. 3-8, three tibial sizing guides 12, 14, 16 are illustrated. Tibial sizing guide A 12, for example, defines first and second opposed planar sides 20, 22, denoted left and right. FIG. 3 shows the left side 20, which may be marked with the word LEFT 24, or with other suitable indicia to indicate that this side should be faced upward when sizing a left tibia bone. FIG. 4 shows the right side 20, which may be marked with the word RIGHT 26, or with other suitable indicia to indicate that this side should be faced upward when sizing a right tibia bone. Thus, the same sizing guide 12 can be used for sizing a left or right tibia bone merely by flipping it over. The sizing guide 12 may be a unitary piece, e.g., of metal, such as stainless steel, or other rigid sterilizable material.

The sizing guide 12 includes a handle 30 and a tibial sizing template 32, which may be integrally formed with the handle, as a unitary piece. The handle 30 optionally defines a ring attachment hole 34 adjacent a first end 36 of the handle. The hole 34 may be generally triangular or trapezoidal, as shown, circular, or other suitable shape. Having a large, e.g., triangular/trapezoidal hole allows for greater relative movement of the tibial sizing guides in use.

A second end 38 of the handle defines a guide opening 40. The guide opening 40 includes one or more longitudinally-extending notches 42, 44, which are used to determine an angle of rotation of the tibial sizing guide 12 when positioned on a prepared tibial bone end.

The tibial sizing template 32 extends from the second end 38 of the handle and includes a medial lobe 50 and a lateral lobe 52 which define a curved indent 54 between them. The medial and lateral lobes 50, 52 are of different dimensions (size and shape), suited to being positioned over the medial and lateral lobes of the tibia bone, as illustrated in the two-way arrows in FIG. 3. For example, the lateral lobe 52 may have a width w₁ at its terminal end 56, which is greater than the corresponding width w₂ of a terminal end 58 of the medial lobe 50. Additionally, the medial lobe may protrude further from the handle than the lateral lobe. The lobes 50, 52 may be marked MEDIAL and LATERAL or with other suitable indicia.

The curved indent 54 is shaped to accommodate the posterior cruciate ligament (PCL), if it is left intact at the discretion of the surgeon. The indent is open at a distal end thereof.

The sizing guides 14 and 16 may be similarly formed to sizing guide 12, except in that each template 32 is of different dimensions (different in size and/or shape to the other templates). Thus, the handles of the sizing guides 12, 14, 16 may be identical.

Each template 32 is associated with a respective tibial component (not shown). Thus, if the template 32 of sizing guide B 14 is determined to be the closest fit to a given left tibia bone when positioned with its left side uppermost, then an appropriate left tibial component (e.g., also labelled B) can be unwrapped from an individual sterilized pouch and fitted to the tibia bone in place of the sizing guide.

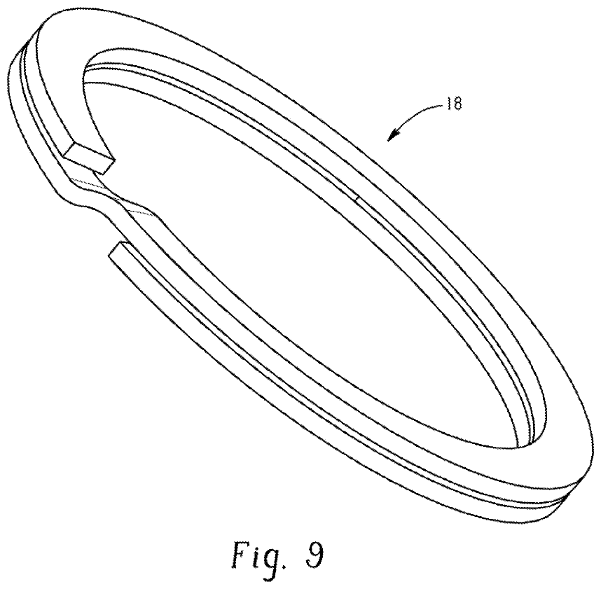

An exemplary ring 18, illustrated in FIG. 9, allows each tibial sizing template 32 to be freely moveable and maneuverable such that it can be placed on the tibial cut surface to evaluate tibial sizing plate size and rotation.

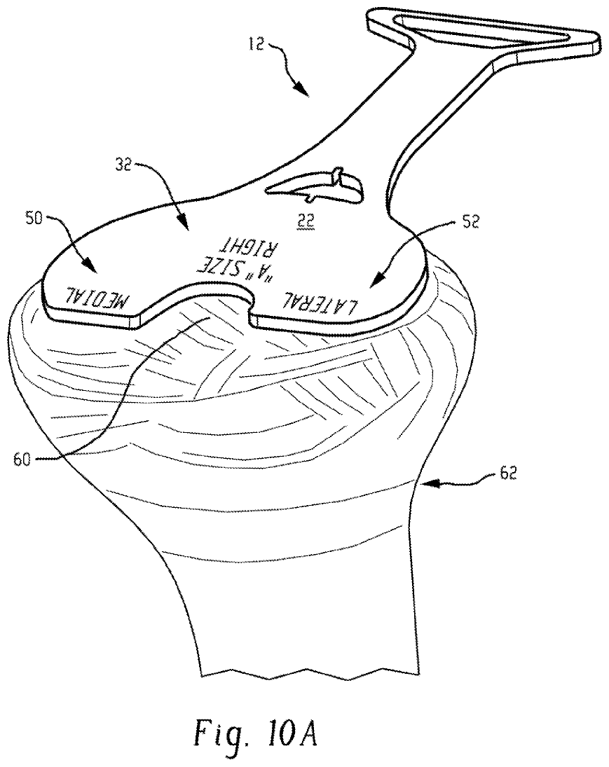

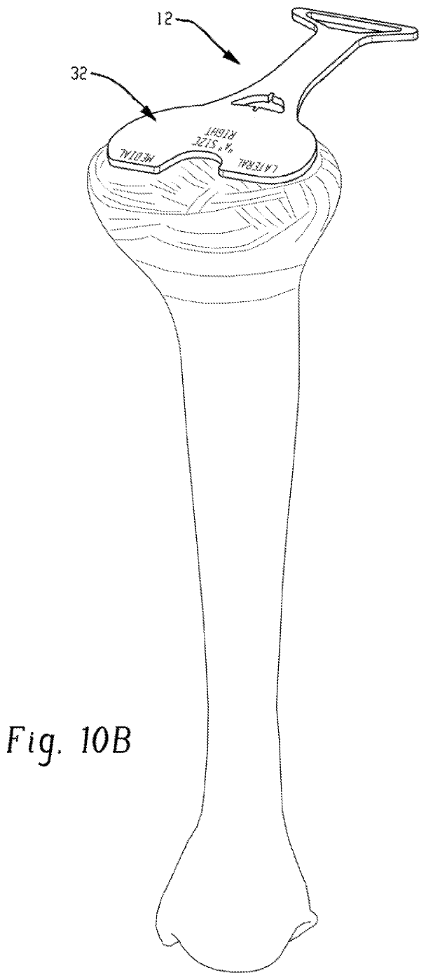

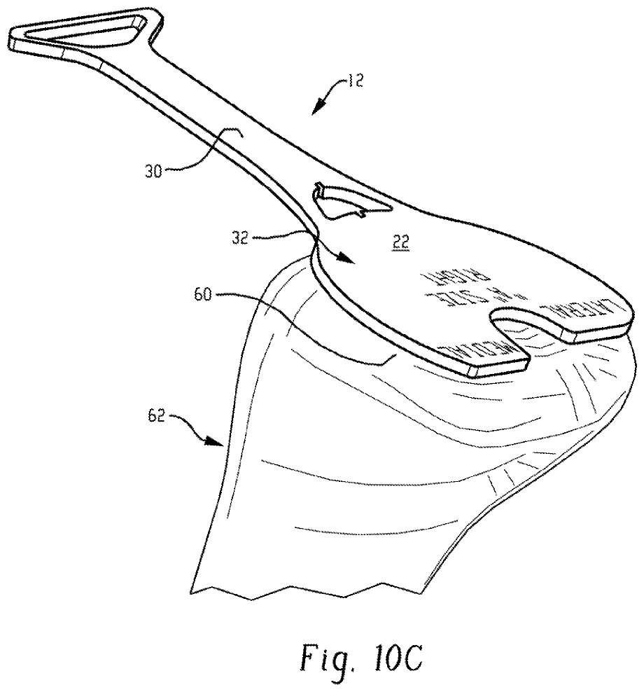

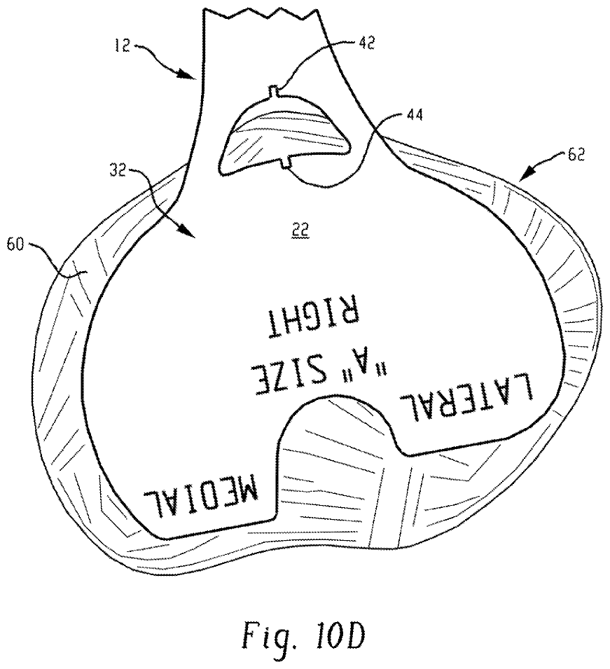

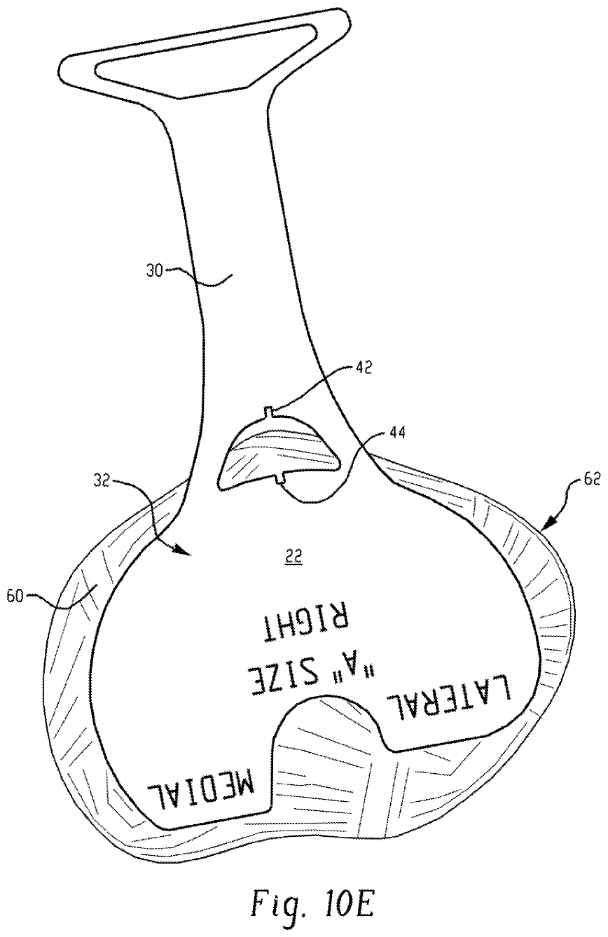

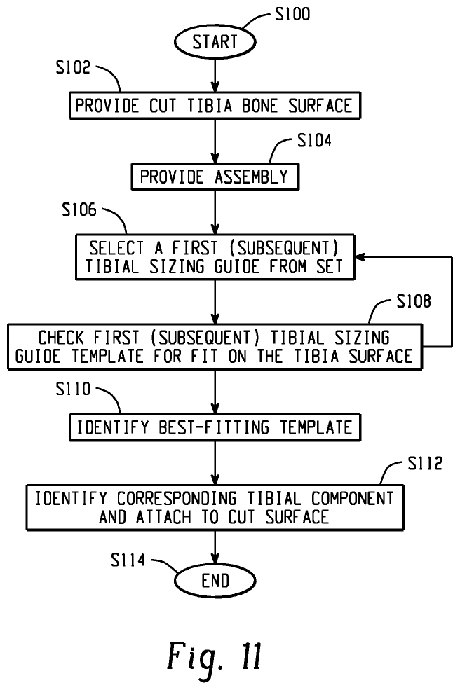

FIGS. 10A-10E illustrates a tibial sizing guide 12 being positioned on a surface 60 of a tibia 62. The ring and other tibial sizing guides are omitted for ease of illustration. During surgery, the surface 60 is typically cut such that the template 32 of the tibial sizing guide lies flush with the surface.

FIG. 11 illustrates a method of use of the assembly 10. The method begins at S100.

At S102, a patient's tibia bone, with its cut surface exposed, is provided, e.g., in a surgical setting.

At S104, an assembly 10 is provided.

At S106, one of the tibial sizing guides 12, 14, 16 is selected, e.g., by a surgeon.

At S108, the selected tibial sizing guide is placed on the cut surface to determine if the template 32 is a good fit. S106 and S108 may be repeated with one or more of the other tibial sizing guides 12, 14, 16. The tibial sizing guides 12, 14, 16 are not removed from the ring 18 during this process.

At S110 a best-fitting template 32 and its angle of rotation are identified. The etched notch 42, 44 on the anterior aspect of the tibial sizing guide allows the surgeon to mark the rotation of the tibial sizing guide so the placement of the tibial sizing plate can be matched. See, for example, FIGS. 10E and 10F. Rotation of the tibial sizing plate is based on several factors selected by the surgeon. In general, the aim is to have the etched notches 42, 44 align with the medial one third of the tibial tubercle. However, tibial sizing plate rotation can be adjusted for various reasons in order to give the patient a well-positioned, stable total knee replacement.

Having the tibial sizing guides 12, 14, 16 all attached to a common ring 18 allows the surgeon to switch easily between templates, e.g., without the need to search in a sterile tray for the next one. Switching between templates can be performed with one hand, while the surgeon has the other hand free to manipulate the bone. The tibial sizing template is not attached to the bone, but merely held in place, so it can be removed quickly.

At S112, once a tibial sizing template has been identified from the set, a corresponding tibial component, such as a tibial sizing plate, can be removed from individual sterile packaging and affixed to the tibial cut surface with one or more attachment members, such as screws, to allow further preparatory work and trialing prior to placement of the final implants. This avoids the need to evaluate other tibial sizing plates, which can remain in their sterile packaging for later use. Examples of tibial sizing plates which may be used are illustrated in the above-mentioned Zimmer Biomet catalog.

Subsequently, a final tibial component (implant) is attached to the tibia surface with one or more attachment members. The size and shape of the implant is determined based on the selected tibial sizing plate and its fit. The final tibial component may be formed from metal and plastic parts. The plastic part is inserted into the final tibial implant. The other side of the plastic part articulates with the final femoral implant.

In another embodiment, the step of using a of tibial sizing plate to select a suitable implant may be omitted.

The method ends at S114.

As noted above, the design of the tibial sizing guide 12, 14, 16 allows it to function for sizing of either a right or left tibia. The “top” can be used to size a right tibia and the “bottom” can be used for a left tibia.

The exemplary tibial sizing templates can provide the surgeon with all the possible tibial plate sizes that the surgeon may need for the procedure within one assembly. This may significantly cut down on the number of times the surgeon would need to wait for a surgical technician to remove an inappropriate tibial sizing plate and replace it with another one for evaluation. As a result, less time is required to complete the surgery.

The ability to select a single tibial sizing plate, based on the corresponding template 32, avoids the need for preparing sterile trays containing multiple tibial sizing plates and to sterilize all the tibial sizing plates after use, even those which have not been used. Each tibial sizing plate can be separately sterile wrapped, since, in general, only one will need to be opened for a given knee. The costs of sterile processing are difficult to estimate. However, the amount of time, water, cleaning solutions, etc. would all be significantly decreased by the elimination of a large sterile tray for every total knee surgical case.

Conventional sterile trays are generally wrapped in either a sterile paper-based wrapping or a sterile packaging (“peel packs”). The large sterile tray containing all the tibial sizing plates requires a large sterile paper-based wrap. The present method allows tibial sizing plates to be individually wrapped in a small sterile peel pack costing about one tenth of the price of a large wrap. The present assembly and method may thus save a considerable amount in sterile packing materials alone.

Reducing the number of times a surgeon has to turn to a rearwardly-positioned table and have a tibial plate size changed can decrease wasted movements by the surgeon and, in turn, reduce the physical wear on the surgeon's body. The prevalence of work-related musculoskeletal disorders among surgeons and interventionalists has been found to be high, as noted in Epstein, et al., “Prevalence of Work-Related Musculoskeletal Disorders Among Surgeons and Interventionalists: A Systematic Review and Meta-analysis,” JAMA Surg. 153(2):e174947 (2018).

It will be appreciated that variants of the above-disclosed and other features and functions, or alternatives thereof, may be combined into many other different systems or applications. Various presently unforeseen or unanticipated alternatives, modifications, variations or improvements therein may be subsequently made by those skilled in the art which are also intended to be encompassed by the following claims.

What is claimed is:

1. An assembly comprising:
a plurality of tibial sizing guides, each of the tibial sizing guides comprising a tibial sizing template, each tibial sizing template being of different dimensions.

2. The assembly of claim 1, wherein the tibial sizing guides are connected.

3. The assembly of claim 2, wherein the tibial sizing guides are connected to a ring.

4. The assembly of claim 1, wherein the tibial sizing guides each include a handle attached to a respective tibial sizing template.

5. The assembly of claim 4, wherein the handle and the respective tibial sizing template are integrally formed.

6. The assembly of claim 4, wherein the handle defines a ring attachment hole.

7. The assembly of claim 1, wherein the tibial sizing guides each include a notch for determining a rotational angle of the tibial sizing template when positioned on a tibia.

8. The assembly of claim 1, wherein each tibial sizing template comprises first and second lobes spaced by an indent.

9. The assembly of claim 8, wherein the first lobe has different dimensions to the second lobe.

10. The assembly of claim 1, wherein the plurality of tibial sizing guides comprises at least three tibial sizing guides.

11. The assembly of claim 1, wherein the template of each of the tibial sizing guides includes opposed planar surfaces.

12. The assembly of claim 11, wherein each template is configured for sizing a left tibia when a first of the opposed planar surfaces is uppermost and for sizing a right tibia surface when a second of the opposed planar surfaces is uppermost.

13. The assembly of claim 12, wherein each template includes markings indicating the medial and lateral sides of a tibia surface when the template is positioned on the tibia surface.

14. The assembly of claim 1, further comprising a plurality of tibial sizing plates, each of the tibial sizing plates corresponding to a respective template of one of the tibial sizing guides.

15. A method of sizing a tibia surface comprising:
providing the assembly of claim 1;
placing one of the tibial sizing guides on a cut surface of a tibia;
placing another of the tibial sizing guides on the cut surface of the tibia;
identifying one of the placed tibial sizing guides as being a best fit for the tibia surface;
identifying a tibial component corresponding to the identified one of the placed tibial sizing guides from a plurality of tibial components of different dimensions; and
fitting the identified tibial component to the tibia surface.

16. The method of claim 15, further comprising determining an angle of rotation of the template using a notch defined in the identified tibial sizing guide.

17. The method of claim 15, wherein each of the plurality of tibial components is individually wrapped.

18. The method of claim 15, wherein the fitting of the identified tibial component to the tibia surface comprises attaching the tibial component with one or more attachment members.

19. A tibial sizing assembly comprising:
a plurality of tibial sizing guides, each of the tibial sizing guides comprising a handle connected to a tibial sizing template, each tibial sizing template being of different dimensions and including first and second lobes of different dimensions to each other, the handle defining a hole; and
a ring, the tibial sizing guides being attached to the ring by their respective holes.
